(19) United States
(12) Patent Application Publication
(10) Pub. No.: US 2011/0033444 A1
(43) Pub. Date: Feb. 10, 2011

(54) Genetic variants in a hypertension susceptibility gene Stk39 and uses thereof

(76) Inventors: Yen-Pei Chang, Baltimore, MD (US); Alan R. Shuldiner, Columbia, MD (US); Braxton D. Mitchell, Towson, MD (US); Patrick F. McArdle, Baltimore, MD (US); Ying Wang, Columbia, MD (US); Sarah E. Dorff, Columbia, MD (US)

Correspondence Address: Benjamin Aaron Adler;ADLER & ASSOCIATES, 8011 Candle Lane, Houston, TX, 77071, US

(21) Appl. No.: 12/925,264

(22) Filed: Oct. 18, 2010

Related U.S. Application Data
(63) Continuation of application No. PCT/US09/02438, filed on Apr. 17, 2009.
(60) Provisional application No. 61/124,671, filed on Apr. 18, 2008.

(51) Int. Cl. A61K 39/395; A61P 9/12; A61K 38/45; C12Q 1/68; C40B 30/04

(52) U.S. Cl. 424/130.1; 424/94.5; 435/6; 506/9

(57) ABSTRACT

The present invention is drawn to diagnosis and treatment of essential hypertension. In this regard, the present invention discloses genetic variants in a hypertension susceptibility gene Stk39 and its use in the diagnosis and treatment of essential hypertension.

### CROSS-REFERENCE TO RELATED APPLICATIONS

This continuation application claims benefit of priority under U.S.C. §120 of international application PCT/US09/002,438, filed Apr. 17, 2009, which claims benefit of priority under 35 U.S.C. §119(e) of provisional application U.S. Ser. No. 61/124,671, filed Apr. 18, 2008, now abandoned, the contents of both of which hereby are incorporated by reference.

### FEDERAL FUNDING LEGEND

This invention was produced using funds obtained through a National Institutes of Health grants (DK54261-07, HL076768-02, HL72515-01, HL088120 and DK32839). Consequently, the Federal government has certain rights in this invention.

### BACKGROUND OF THE INVENTION

1. Field of the Invention

The present invention relates generally to the fields of molecular biology and genetics. More specifically, the present invention discloses a gene associated with blood pressure levels and hypertension status and its use as a marker in the diagnosis, as a target for medication and to predict the effect of drugs used in the treatment of hypertension.

2. Description of the Related Art

Hypertension is a leading cause of mortality and morbidity worldwide, contributing to cardiovascular disease, stroke, and end stage kidney disease. The most common form, essential hypertension (EH), is widely believed to involve multiple genes with variant alleles. Like other complex disorders, manifestation of essential hypertension in any one individual is likely dependent on different genetic and environment factors, making identification of essential hypertension susceptibility genes in the general population a major challenge. Studies on the genetic basis of rare monogenetic blood pressure disorders, by contrast, have identified mutations in genes affecting a single physiologic pathway, kidney salt transport. While such studies highlight the importance of this pathway to blood pressure regulation, the underlying genetic basis for essential hypertension remains poorly understood.

Despite the knowledge of pathways important in the regulation of blood pressure, the prior art is deficient in the underlying genetic basis for essential hypertension. Furthermore, the prior art is also deficient in a marker that can be used to diagnose essential hypertension, used as a target for medication and used to predict the effect of a drug that is used to treat hypertension. The current invention fulfils this long standing need in the art.

### SUMMARY OF THE INVENTION

In one embodiment of the present invention, there is provided a method for determining susceptibility of an individual to develop essential hypertension. This method comprises detecting at least one single nucleotide polymorphism in the Stk39 gene in a sample from the individual, wherein the presence of the single nucleotide polymorphism in the Stk39 gene in the sample indicates that the individual is susceptible to develop essential hypertension.

In another embodiment of the present invention, there is provided a kit. Such a kit comprises one or more Stk39 single nucleotide polymorphism-specific oligonucleotide sequences; and reagents to detect binding of the sequences. In yet another embodiment of the present invention, there is provided a kit. Such a kit comprises an antibody specific to a peptide encoded by at least one Stk39 single nucleotide polymorphism; and reagents to detect binding of the antibody.

In yet another embodiment of the present invention, there is provided a method of treating an individual with essential hypertension. Such a method comprises administering pharmacologically effective amounts of a compound that inhibits at least one single nucleotide polymorphism in Stk39 at the nucleic acid level and/or inhibits at least one protein encoded by a single nucleotide polymorphism in Stk39 to the individual, thereby treating the individual with essential hypertension.

In another embodiment of the present invention, there is provided a method of treating an individual with essential hypertension. Such a method comprises administering pharmacologically effective amounts of an anti-hypertensive drug to the individual, wherein the individual has at least one single nucleotide polymorphism in Stk39 gene, thereby treating the individual with essential hypertension.

In yet another embodiment of the present invention, there is provided a method of treating an individual with essential hypertension. Such a method comprises reducing sodium intake in the individual, where the individual has at least one single nucleotide polymorphism in Stk39 gene, thereby treating the individual with essential hypertension.

In still yet another embodiment of the present invention, there is provided a method for screening compound(s) useful in the treatment of essential hypertension. This method comprises contacting a cell comprising at least one single nucleotide polymorphism in Stk39 with the compound(s). The detection of the single nucleotide polymorphism in the presence of the compound(s) is then compared with the detection of the single nucleotide polymorphism in the absence of said compound(s), where no detection or decreased level of detection of the single nucleotide polymorphism in the Stk39 gene in the cell in the presence of the compound(s) compared to detection in the absence of the compound(s) indicates that the compound(s) is useful in the treatment of essential hypertension.

In another embodiment of the present invention, there is provided a transgenic mouse. Such a mouse comprises a mouse Stk39 gene with one or more single nucleotide polymorphism or a human Stk39 gene with one or more single nucleotide polymorphism. The single nucleotide polymorphism(s) in the Stk39 gene results in complete or partial inactivation of the mouse or the human Stk39 gene further related embodiment of the present invention, there is provided a method of determining genotype-specific response to a compound useful in the treatment of hypertension. Such a method comprises administering a test or candidate compound to the transgenic mouse described supra and comparing the blood pressure in the mouse in the presence of the compound and in the absence of the compound. A lowering of blood pressure in the presence of the compound compared to blood pressure in the absence of the compound indicates that an individual carrying this genotype will respond favorably to the compound, i.e., lower the blood pressure in that individual.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and do not limit the scope of the invention.

FIGS. 1A-1C shows results from genomewide association (GWA) analysis of the Old Order Amish population. FIG. 1A shows whole genome association scan results for SBP in 551 AFDS subjects. Significance levels (Ps) were adjusted for family structure. FIG. 1B shows exonic structure, association between STK39 SNPs and SBP/DBP. FIG. 1C shows LD relationship between SNPs. The location of associated SNPs in LD bin 1 and 2 are denoted by black and gray lines, respectively. Pairwise LD relationships (R²) between SNPs indicated by color (black=1, white=0, shades of gray=0<R²<1) are shown.

FIGS. 2A-2E show immunolocalization of SPAK in kidney. FIG. 2A shows that SPAK strongly localizes to the thick ascending limb (TAL) of the loop of Henle and the cortical collecting duct (CCD). FIG. 2B shows immunolocalization of aquaporin 2 (AQP2) in the same section as FIG. 2A as a segment-specific marker for CCD. FIG. 2C shows immunolocalization of SPAK in distal convoluted tubules (DCT). FIG. 2D shows immunolocalization of NCC as a segment-specific marker for DCT. FIG. 2E shows that immunolabeling is specific by incubation of the antibody with the immunizing peptide.

FIGS. 3A-3B show results of sequence analysis of 8 exons and conserved intronic elements (CEs) in the 76 kb region of STK39 containing SNPs associated with blood pressure. FIG. 3A shows human-dog and human-mouse (top) sequence alignment of the 76 kb region associated with blood pressure. Conserved elements (CEs) are defined as intronic elements with as much sequence identity across species as coding exons. Blue and red peaks denote conserved intronic and exonic regions, respectively with greater than >90% sequence identity. The locations of conserved elements 3 and 5 (CE3 and CE5) are shown above the alignment. FIG. 3B shows results of the luciferase assay in HeLa cells showing that both CE3 and CE5 possess suppressor activity.

### DETAILED DESCRIPTION OF THE INVENTION

### I. Definitions

The use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

The use of the term “contacting” is used to refers to any suitable method of bringing the potential hypertension medication described herein into contact with a cell that has one or more variants of STK39. In vitro or ex vivo this is achieved by exposing the cell to the medication in a suitable medium. For in vivo applications, any method of administration is suitable as described herein.

### II. Present Invention

The present invention is directed to a method for determining susceptibility of an individual to developing essential hypertension, comprising: detecting at least one single nucleotide polymorphism in the in Stk39 gene in a sample from the individual, wherein the presence of the single nucleotide polymorphism in the sample indicates that the individual is susceptible to develop essential hypertension. Additionally, the single nucleotide polymorphism(s) may be located in intron 10 of the STK39 gene. Representative examples of the single nucleotide polymorphism may include rs2278785, rs3769394, rs10497336, rs3769392, rs3754781, rs3754777, rs1400644, rs1400645, rs10497337, rs6749447, rs6734514, rs4668044, rs2063959, rs10497331, rs10497332, rs9287890, rs10497333, rs10497334, rs4667995, rs1448833, rs4667556, rs6728405, rs6745588, rs755844, rs10497335, rs1517343, rs6740826, rs10497338, rs950535, rs4667996, rs1816977, rs4668021, rs2138753, rs13419175, rs1448831, rs11685807, rs6718607, rs4668002, rs6433032, rs4233815, rs6433027, rs4668000, rs4667548, rs9789702, rs4667551, rs10170500, rs3754776, rs6740826, rs16855116, rs1685513, rs6714609, rs7589259, rs4668046, rs1517329, rs2063958, rs4668040, rs13385577, rs10202854, rs4667570, rs7605161, rs1400641, rs3769393, rs16855027, rs16855079, rs12692877, or rs35929607.

The single nucleotide polymorphism may be detected at the nucleic acid or protein level. Assays used to detect the single nucleotide polymorphism at the nucleic acid level may include but is not limited to a DNA microarray, a PCR assay or FISH and those used to detect the single nucleotide polymorphism at the protein level may include but is not limited to ELISA, Western blot, Immunohistochemistry, or HPLC. Additionally, the biological sample may include but is not limited to serum, urine, skin biopsy or buccal swab. Furthermore, the presence of the single nucleotide polymorphism in the sample indicate that the individual can be treated with an anti-hypertensive drug with a decreased adverse effect on glucose homeostasis, a low salt diet with a decreased adverse effect on glucose homeostasis, or both. Examples of the anti-hypertensive drug may include but is not limited to a diuretic, an angiotensin converting enzyme inhibitor, angiotensin II receptor antagonist, an alpha blocker, a beta blocker, a calcium channel blocker or a direct renin inhibitor.

The present invention is also directed to a kit, comprising: one or more Stk39 single nucleotide polymorphism-specific oligonucleotide sequences. Additionally, the kit may also comprise reagents to detect binding the sequences. Since kits comprising oligonucleotide sequences are known in the art, one of skill in the art would know the reagents that are included in this kit to enable detection of binding of the oligonucleotide sequences. Further, the position of the single nucleotide polymorphisms and the type of single nucleotide polymorphism is the same as discussed supra.

The present invention is further directed to a kit, comprising an antibody specific to a peptide encoded by at least one Stk39 single nucleotide polymorphism, and reagents to detect binding of said antibody. Since kits comprising antibodies are known in the art, one of skill in the art would know the reagents that are included in this kit to enable detection of binding of the antibodies. Further, the position of the single nucleotide polymorphisms and the type of single nucleotide polymorphism is the same as discussed supra.

The present invention is also directed to a method of treating an individual with essential hypertension, comprising: administering pharmacologically effective amounts of a compound that inhibits at least one single nucleotide polymorphism in Stk39 at the nucleic acid level and/or inhibits at least one protein encoded by a single nucleotide polymorphism in Stk39 to the individual, thereby treating the individual with essential hypertension. This method may further comprise reducing sodium intake in the individual. Additionally, the administration of the compound may result in a decreased adverse effect on the glucose homeostasis of the individual. Further, the position and type of the single nucleotide polymorphism are the same as discussed supra.

The present invention is further directed to a method of treating an individual with essential hypertension, comprising: administering pharmacologically effective amounts of an anti-hypertensive drug to the individual, where the individual has at least one single nucleotide polymorphism in the Stk39 gene, thereby treating the individual with essential hypertension. This method may further comprise reducing the sodium intake in said individual. Additionally, the administration of the anti-hypertensive drug may result in decreased adverse effect on glucose homeostasis of the individual. Examples of the anti-hypertensive drug may include but is not limited to a diuretic, an angiotensin converting enzyme inhibitor, an angiotensin II receptor antagonist, an alpha blocker, a beta blocker, a calcium channel blocker or a direct renin inhibitor. Further, the position and types of single nucleotide polymorphism are as discussed supra.

The present invention is still further directed to a method of treating an individual with essential hypertension, comprising: reducing sodium intake in the individual, where the individual has at least one single nucleotide polymorphism in the Stk39 gene, thereby treating the individual with essential hypertension. This method may further comprise administering an anti-hypertensive drug, a compound that inhibits at least one single nucleotide polymorphism in Stk39 at nucleic acid level, inhibits at least one protein encoded by a single nucleotide polymorphism in Stk39 or a combination thereof. Further, administration of the anti-hypertensive drug or the compound may result in decreased adverse effect on the glucose homeostasis of the individual. Examples of the anti-hypertensive drugs and position and types of single nucleotide polymorphism are as discussed supra.

The present invention is further directed to a method for screening compound(s) useful in the treatment of essential hypertension, comprising: contacting a cell comprising at least one single nucleotide polymorphism in Stk39 with the compound(s), and comparing detection of the single nucleotide polymorphism in the presence of the compound(s) and in the absence of the compound(s), where no detection or decreased level of detection of the single nucleotide polymorphism in the cell in the presence of the compound(s) compared to detection in the absence of the compound(s) indicates that the compound(s) is useful in the treatment of essential hypertension. The cell with the single nucleotide polymorphism may be in vitro or in vivo. The location and type of single nucleotide polymorphisms, the type of detection and the assays used in this method are as discussed supra. The cell examined in such a method may be a renal cell.

The present invention is further directed to a method of determining whether blood pressure of an individual would decrease after reducing dietary sodium intake in said individual, comprising: detecting at least one single nucleotide polymorphism in the Stk39 gene in a sample from the hypersensitive individual, wherein the presence of said single nucleotide polymorphism in the sample indicates that the blood pressure of the individual would decrease after reducing dietary sodium intake. The location and type of the single nucleotide polymorphism in the Stk39 gene, the type of detection of these polymorphisms, the type of sample analyzed and the methods to detect these polymorphisms are the same as discussed supra.

The present invention is also directed to a transgenic mouse comprising a mouse Stk39 gene with one or more single nucleotide polymorphism or a human Stk39 gene with one or more single nucleotide polymorphism, where presence of the single nucleotide polymorphism results in complete or partial inactivation of the mouse or human Stk39 gene. The location and type of these single nucleotide polymorphisms are the same as discussed supra.

The present invention is further directed to a method of determining a genotype-specific response to a compound useful in the treatment of hypertension, comprising administering said compound to the transgenic mouse described supra and comparing the blood pressure of the mouse in the presence of the compound and in the absence of the compound, where a decrease or lowering of the blood pressure in the presence of the compound compared to the blood pressure in the absence of the compound indicates that an animal having the genotype will respond favorably to the compound, i.e., the compound results in a decrease in blood pressure in the animal.

The present invention discloses that genetic variants in a hypertension susceptibility gene Stk39 can predict efficacy of anti-hypertension medication and salt reduction on blood pressure control. Briefly, the present invention used a screening approach that systematically examined 100,000 genetic markers in the entire genome and identified a novel gene associated with blood pressure levels and hypertension status. This gene named Stk39, encodes a serine-threonine kinase and there is emerging evidence that this kinase plays a role in renal salt transport. However, the role of this gene in essential hypertension has not been examined so far. The present invention, for the first time, examined the common variants in the Stk39 gene and established its role in essential hypertension. The data presented herein shows that Stk39 genotypes are associated with salt sensitivity and other metabolic traits.

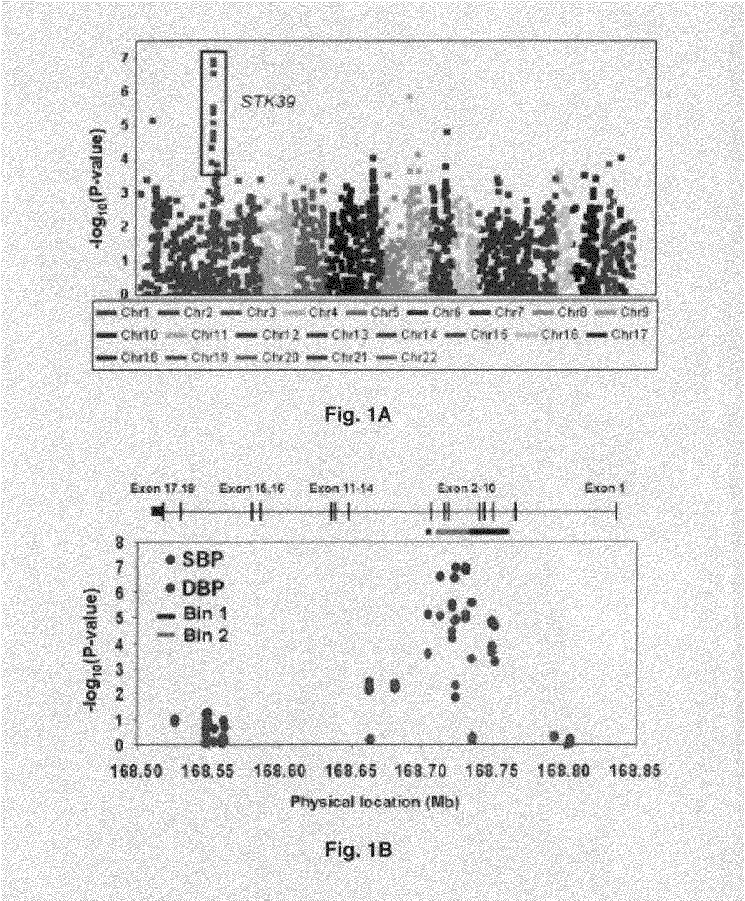

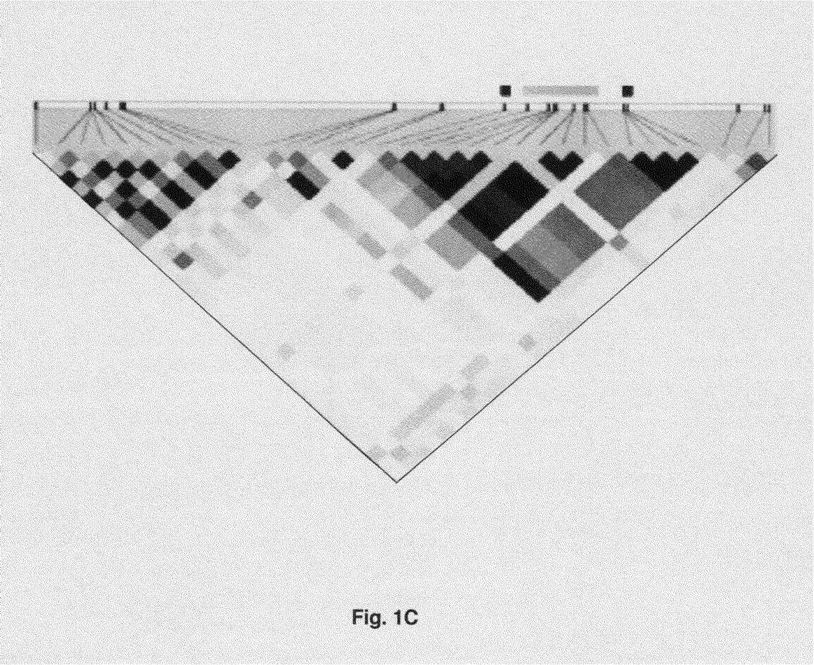

To identify common EH susceptibility genes, a genomewide association (GWA) analysis was conducted in the Old Order Amish, a closed founder population who emigrated to the Lancaster, Pa. area from Switzerland in the early 1700's. The members of this community are genetically homogenous and share a relatively homogeneous lifestyle, making this population ideal for identifying genes that underlie complex diseases. Genotype data from Affymetrix GeneChip Human Mapping 100K platform and SBP/DBP measurements were obtained in 551 subjects of the Amish Family Diabetes Study (4) (AFDS, Table 1A). The most significant GWA for SBP was with a cluster of SNPs located on chromosome 2q24.3 (FIG. 1A, P=1.1×10⁻⁷ to 2.2×10⁻⁵). All association signals are available online and those with P<0.0005 are provided in Table 1B. In Table 1B, the position and associated loci of SNPs are based on NCBI dbSNP build 128. The MAFs, genotype call rate and Ps for HWE are calculated based on the original 551 AFDS samples used in GWA. Ps for SBP and DBP are based on age and sex adjusted regression analysis performed using a variance component approach to account for family structure.

TABLE 1A
Characteristics of Amish Study subjects

| | Initial screening | | |
|---|---|---|---|
| | sample | Expanded sample | Replication sample |
| | AFDS* | AFDS* | HAPI |
| N | 551 | 1,093 | 868 |
| Age (yrs) | 54 ± 13.1 | 44.2 ± 19.3 | 43.8 ± 14.0 |
| Sex (% male) | 44% | 46% | 53% |
| BMI (kg/m²) | 28.1 ± 5.2 | 27.1 ± 04.9 | 26.6 ± 4.5 |
| SBP (mmHg) | 127.1 ± 19.6 | 123.7 ± 17.6 | 121.1 ± 14 |
| DBP (mmHg) | 80.4 ± 10.2 | 78.2 ± 9.8 | 76.6 ± 8.7 |
| % on | 11.0% | 9.3% | 0.2% |
| BP-lowering | | | |
| medication | | | |

Only 124 subjects (22.5%) in the initial AFDS sample and 148 subjects (13.5%) of the expanded AFDS sample had type 2 diabetes.
**HAPI subject who were on BP-lowering medication had these medications discounted.

TABLE 1B
GWA signals with P < 0.0005

| Reference | | | | | Call | | | |
|---|---|---|---|---|---|---|---|---|
| SNP# | Cytogenetic | Position | Loci | MAF | rate | HWE | Trait | P-value |
| rs4927069 | 1p33 | 48035206 | LOC388630 | 0.29 | 0.98 | 0.03 | SBP | 3.60E−04 |
| rs7534158 | 1p31.1 | 76960980 | | 0.06 | 0.99 | 0.85 | SBP | 6.30E−06 |
| rs1722663 | 2q24.2 | 161529051 | | 0.05 | 0.99 | 0.02 | SBP | 1.07E−04 |
| rs1722662 | 2q24.2 | 161529455 | | 0.05 | 1.00 | 0.02 | SBP | 4.17E−05 |
| rs2278785 | 2q24.3 | 168705021 | STK39 | 0.19 | 1.00 | 0.89 | SBP | 7.52E−06 |
| rs3769394 | 2q24.3 | 168713624 | STK39 | 0.09 | 0.99 | 0.84 | SBP | 2.59E−07 |
| rs10497336 | 2q24.3 | 168721411 | STK39 | 0.08 | 0.95 | 0.97 | SBP | 3.01E−06 |
| rs3769392 | 2q24.3 | 168721920 | STK39 | 0.10 | 0.90 | 0.71 | SBP | 3.73E−06 |
| rs3754781 | 2q24.3 | 168723722 | STK39 | 0.10 | 0.99 | 0.99 | SBP | 2.66E−07 |
| rs3754777 | 2q24.3 | 168724160 | STK39 | 0.09 | 0.99 | 0.84 | SBP | 1.08E−07 |
| rs1400644 | 2q24.3 | 168730859 | STK39 | 0.09 | 1.00 | 0.75 | SBP | 1.06E−07 |
| rs1400645 | 2q24.3 | 168731227 | STK39 | 0.09 | 1.00 | 0.78 | SBP | 1.32E−07 |
| rs10497337 | 2q24.3 | 168735395 | STK39 | 0.09 | 0.99 | 0.46 | SBP | 2.62E−06 |
| rs6749447 | 2q24.3 | 168749632 | STK39 | 0.19 | 1.00 | 0.85 | SBP | 1.47E−05 |
| rs6734514 | 2q24.3 | 168749805 | STK39 | 0.19 | 0.99 | 0.98 | SBP | 1.41E−05 |
| rs4668044 | 2q24.3 | 168750016 | STK39 | 0.19 | 0.98 | 0.74 | SBP | 1.83E−05 |
| rs2063959 | 2q24.3 | 168751169 | STK39 | 0.19 | 0.98 | 0.96 | SBP | 2.20E−05 |
| rs1157535 | 2q31.3 | 181069981 | | 0.23 | 0.97 | 0.46 | SBP | 3.87E−04 |
| rs1358520 | 2q31.3 | 181148326 | | 0.23 | 0.96 | 0.68 | SBP | 3.81E−04 |
| rs736019 | 2q31.3 | 181234813 | | 0.25 | 0.99 | 0.92 | SBP | 4.23E−04 |
| rs2063452 | 2q32.3 | 195720297 | | 0.24 | 1.00 | 0.10 | SBP | 1.34E−04 |
| rs2130343 | 2q32.3 | 195720328 | | 0.24 | 0.98 | 0.26 | SBP | 3.06E−04 |
| rs10497749 | 2q32.3 | 195726289 | | 0.23 | 1.00 | 0.09 | SBP | 2.45E−04 |
| rs6736088 | 2q32.3 | 195726614 | | 0.24 | 1.00 | 0.10 | SBP | 1.34E−04 |
| rs1489771 | 2q32.3 | 195843043 | | 0.22 | 0.97 | 0.04 | SBP | 3.12E−04 |
| rs2037597 | 2q32.3 | 195844798 | | 0.23 | 1.00 | 0.23 | SBP | 2.24E−04 |
| rs9288243 | 2q32.3 | 195857142 | | 0.23 | 0.99 | 0.58 | SBP | 3.07E−04 |
| rs1489769 | 2q32.3 | 195873229 | | 0.24 | 1.00 | 0.29 | SBP | 1.30E−04 |
| rs10511046 | 3p12.3 | 76616787 | | 0.12 | 1.00 | 0.05 | SBP | 3.66E−04 |
| rs3804138 | 3q29 | 197292085 | TFRC | 0.05 | 0.93 | 0.12 | SBP | 3.47E−04 |
| rs10520303 | 4q34.2 | 176855847 | GPM6A | 0.13 | 0.99 | 0.06 | SBP | 4.21E−04 |
| rs1862168 | 5q34 | 163127599 | | 0.24 | 0.99 | 0.23 | SBP | 3.32E−04 |
| rs6864913 | 5q34 | 164840549 | | 0.37 | 0.94 | 0.19 | SBP | 2.66E−04 |
| rs916690 | 7q31.1 | 108678221 | | 0.35 | 0.97 | 0.72 | SBP | 2.11E−04 |
| rs2188445 | 7q31.1 | 108690421 | | 0.35 | 1.00 | 0.79 | SBP | 3.38E−04 |
| rs10487512 | 7q31.1 | 108700289 | | 0.35 | 1.00 | 0.82 | SBP | 3.19E−04 |
| rs980400 | 7q31.1 | 108704453 | | 0.35 | 1.00 | 0.85 | SBP | 3.13E−04 |
| rs2106444 | 7q31.1 | 108714249 | | 0.35 | 0.97 | 0.53 | SBP | 7.84E−05 |
| rs7000419 | 8p11.21 | 40986119 | LOC727725 | 0.12 | 0.98 | 0.60 | SBP | 2.65E−04 |
| rs10511730 | 9p21.3 | 23553772 | | 0.10 | 1.00 | 0.81 | SBP | 2.00E−04 |
| rs4977950 | 9p21.3 | 24722482 | | 0.13 | 0.94 | 0.53 | SBP | 1.22E−06 |
| rs10511984 | 9q21.11 | 72360115 | TRPM3 | 0.36 | 0.97 | 0.42 | SBP | 6.56E−05 |
| rs10521467 | 9q21.13 | 77841311 | PCSK5 | 0.22 | 1.00 | 0.72 | SBP | 1.96E−04 |
| rs4934391 | 10q23.31 | 90038129 | C10orf59 | 0.28 | 1.00 | 0.44 | SBP | 4.08E−04 |
| rs1035796 | 10q23.31 | 90041677 | C10orf59 | 0.28 | 0.96 | 0.15 | SBP | 1.39E−04 |
| rs10509589 | 10q23.31 | 91804145 | | 0.11 | 0.97 | 0.45 | SBP | 1.36E−05 |
| rs1269790 | 12q24.21 | 113160378 | | 0.09 | 0.98 | 0.06 | SBP | 3.25E−04 |
| rs4130296 | 12q24.22 | 115870468 | FBXW8 | 0.09 | 1.00 | 0.27 | SBP | 4.45E−04 |
| rs2639187 | 15q26.2 | 94204314 | | 0.17 | 1.00 | 0.16 | SBP | 3.33E−04 |
| rs163234 | 16p12.3 | 20559524 | ACSM1 | 0.27 | 1.00 | 0.14 | SBP | 2.07E−04 |
| rs2022174 | 16p12.1 | 24164733 | | 0.14 | 1.00 | 0.17 | SBP | 2.52E−04 |
| rs10521133 | 16p12.1 | 26186926 | | 0.33 | 0.99 | 0.68 | SBP | 2.60E−04 |
| rs10521134 | 16p12.1 | 26187725 | | 0.33 | 0.99 | 0.59 | SBP | 2.92E−04 |
| rs10521135 | 16p12.1 | 26189217 | | 0.33 | 1.00 | 0.64 | SBP | 3.90E−04 |
| rs621034 | 18p11.23 | 8457794 | | 0.11 | 0.98 | 0.76 | SBP | 2.69E−04 |
| rs1588112 | 18q23 | 71838706 | | 0.05 | 0.99 | 0.18 | SBP | 3.22E−04 |
| rs10485747 | 20p12.2 | 11253191 | | 0.28 | 0.94 | 0.57 | SBP | 1.24E−04 |
| rs10482900 | 21q21.1 | 19468467 | | 0.43 | 1.00 | 0.81 | SBP | 8.22E−05 |
| rs10493881 | 1p21.3 | 95200639 | LOC729970 | 0.21 | 1.00 | 0.67 | DBP | 3.60E−04 |
| rs1386821 | 1q21.3 | 152648673 | IL6R | 0.23 | 0.99 | 0.03 | DBP | 1.32E−04 |
| rs2278785 | 2q24.3 | 168705021 | STK39 | 0.19 | 1.00 | 0.89 | DBP | 2.66E−04 |
| rs3769394 | 2q24.3 | 168713624 | STK39 | 0.09 | 0.99 | 0.84 | DBP | 9.09E−06 |
| rs10497336 | 2q24.3 | 168721411 | STK39 | 0.08 | 0.95 | 0.97 | DBP | 6.75E−05 |
| rs3769392 | 2q24.3 | 168721920 | STK39 | 0.10 | 0.90 | 0.71 | DBP | 3.59E−05 |
| rs3754781 | 2q24.3 | 168723722 | STK39 | 0.10 | 0.99 | 0.99 | DBP | 1.46E−05 |
| rs3754777 | 2q24.3 | 168724160 | STK39 | 0.09 | 0.99 | 0.84 | DBP | 1.29E−05 |
| rs1400644 | 2q24.3 | 168730859 | STK39 | 0.09 | 1.00 | 0.75 | DBP | 7.34E−06 |
| rs1400645 | 2q24.3 | 168731227 | STK39 | 0.09 | 1.00 | 0.78 | DBP | 1.08E−05 |
| rs10497337 | 2q24.3 | 168735395 | STK39 | 0.09 | 0.99 | 0.46 | DBP | 4.68E−04 |
| rs6749447 | 2q24.3 | 168749632 | STK39 | 0.19 | 1.00 | 0.85 | DBP | 2.43E−04 |
| rs6734514 | 2q24.3 | 168749805 | STK39 | 0.19 | 0.99 | 0.98 | DBP | 1.73E−04 |
| rs4668044 | 2q24.3 | 168750016 | STK39 | 0.19 | 0.98 | 0.74 | DBP | 1.32E−04 |
| rs1157535 | 2q31.3 | 181069981 | | 0.23 | 0.97 | 0.46 | DBP | 8.40E−05 |
| rs736019 | 2q31.3 | 181234813 | | 0.25 | 0.99 | 0.92 | DBP | 3.54E−04 |
| rs3797252 | 5q11.2 | 53536371 | ARL15 | 0.18 | 0.99 | 0.47 | DBP | 1.13E−04 |
| rs10516015 | 5q34 | 165866346 | | 0.06 | 0.97 | 0.11 | DBP | 3.07E−04 |
| rs10499967 | 7q31.1 | 108643734 | | 0.33 | 1.00 | 0.66 | DBP | 1.53E−04 |
| rs916690 | 7q31.1 | 108678221 | | 0.35 | 0.97 | 0.72 | DBP | 3.11E−05 |
| rs2106442 | 7q31.1 | 108687109 | | 0.36 | 1.00 | 0.89 | DBP | 6.56E−05 |
| rs2188445 | 7q31.1 | 108690421 | | 0.35 | 1.00 | 0.79 | DBP | 2.18E−05 |
| rs6944700 | 7q31.1 | 108696569 | | 0.35 | 0.98 | 0.79 | DBP | 6.58E−05 |
| rs10487512 | 7q31.1 | 108700289 | | 0.35 | 1.00 | 0.82 | DBP | 2.20E−05 |
| rs980400 | 7q31.1 | 108704453 | | 0.35 | 1.00 | 0.85 | DBP | 2.27E−05 |
| rs2106444 | 7q31.1 | 108714249 | | 0.35 | 0.97 | 0.53 | DBP | 8.02E−06 |
| rs7000419 | 8p11.21 | 40986119 | LOC727725 | 0.12 | 0.98 | 0.60 | DBP | 2.83E−04 |
| rs4977950 | 9p21.3 | 24722482 | | 0.13 | 0.94 | 0.53 | DBP | 1.98E−04 |
| rs10521467 | 9q21.13 | 77841311 | PCSK5 | 0.22 | 1.00 | 0.72 | DBP | 6.44E−05 |
| rs10509786 | 10q25.1 | 106912345 | SORCS3 | 0.15 | 1.00 | 0.09 | DBP | 3.10E−04 |
| rs10509788 | 10q25.1 | 106919911 | SORCS3 | 0.15 | 1.00 | 0.09 | DBP | 3.10E−04 |
| rs163234 | 16p12.3 | 20559524 | ACSM1 | 0.27 | 1.00 | 0.14 | DBP | 1.36E−04 |
| rs10485747 | 20p12.2 | 11253191 | | 0.28 | 0.94 | 0.57 | DBP | 1.43E−04 |
| rs1735232 | 21q21.1 | 15639813 | | 0.16 | 0.99 | 0.57 | DBP | 6.75E−05 |

The associated SNPs on chromosome 2q24.3 were located within the gene STK39 (serine threonine kinase 39), which encodes SPAK (Ste20-related proline-alanine-rich kinase). A growing body of evidence indicates that SPAK interacts with cation-chloride co-transporters as a part of an evolutionary conserved signaling pathway important for cotransporter (NCC) and bumetanide-sensitive Na-controlling salt transport in osmotic cell volume regulation. In addition to the well known effects of SPAK on Na⁺—K⁺—2Cl⁻-chloride cotransporter (NKCC1), SPAK also phosphorylates the thiazide sensitive, Na⁺—Cl⁻⁺—K⁻-2Cl⁻ cotransporter (NKCC2). Both transporters play major roles in renal salt excretion, as underscored by the direct involvement of NCC and NKCC2 in autosomal recessive conditions of hypotension, hypokalemic metabolic alkalosis and salt wasting. SPAK is phosphorylated by WNK1 and WNK4 (lysine-deficient protein kinase 1 and 4). Rare mutations in these 2 genes lead to autosomal dominant pseudohypoaldosteronism type II characterized by hyperkalemia, hypertension, and sensitivity to thiazide diuretics.

The finding herein, combined with the role of SPAK in renal electrolyte transport, make SPAK a logical EH candidate gene. STK39 contains 18 exons that span approximately 300 kb. There are 39 SNPs on the Affymetrix 100K platform located within 5 kb of STK39. The associations of these SNPs with BP and their pair-wise linkage disequilibrium (LD) relationships are shown in FIGS. 1B and 1C. BP association signals (P<0.0001) are derived from 2 groups of highly correlated SNPs (R²>0.8 within groups and R²>0.4 between groups) with average minor allele frequencies (MAF) of 0.19 and 0.09. One SNP from each group, rs6749447 and rs3754777, in all AFDS subjects with BP measurements (N=1,093) was genotyped herein and the association remained highly significant (P<10⁻⁵, Table 2). For simplicity, SNPs that are highly correlated with either rs6749447 or rs3754777 (R²>0.8) will be referred to as belonging to LD bin 1 and 2, respectively (FIGS. 1B and 1C). For both SNPs, homozygotes for the minor allele had an estimated 5 mmHg higher SBP and 2 mm Hg higher DBP than subjects who were homozygous for the major allele; the association was highly significant when tested using an additive or dominant model. The associations remained significant when the analysis was restricted to subjects not taking BP-lowering medication (P<0.005).

TABLE 2
Genotype-specific SBP and DBP (mm Hg, mean ± SEM) for representative
associated SNPs from LD bin1 and bin 2 of the expanded AFDS subjects.

| | | | | | Model (P) | | |
|---|---|---|---|---|---|---|---|
| SNP ID | trait | Genotype | | | Additive | Dominant | Recessive |
| rs6749447 | | TT | TG | GG | | | |
| LD bin 1 | N. | 698 | 359 | 36 | | | |
| | SBP | 121.7 ± 1.1 | 127.1 ± 1.1 | 127.1 ± 2.9 | 0.00001 | 0.000002 | NS |
| | DBP | 80.2 ± 0.7 | 82.5 ± 0.7 | 83.0 ± 1.7 | 0.001 | 0.0001 | NS |
| | N | 638 | 320 | 32 | | | |
| | SBP* | 120.2 ± 1.0 | 124.4 ± 1.0 | 123.9 ± 2.6 | 0.004 | 0.0001 | NS |
| | DBP* | 79.7 ± 0.6 | 81.8 ± 0.7 | 81.6 ± 1.7 | 0.003 | 0.002 | NS |
| rs3754777 | | GG | GA | AA | | | |
| LD bin 2 | N. | 877 | 203 | 9 | | | |
| | SBP | 122.0 ± 4.8 | 128.7 ± 4.9 | 127.4 ± 4.9 | 0.000005 | 0.000002 | NS |
| | DBP | 80.9 ± 3.3 | 82.6 ± 3.3 | 89..5 ± 3.3 | 0.001 | 0.002 | 0.009 |
| | N. | 801 | 175 | 7 | | | |
| | SBP* | 120.4 ± 4.9 | 125.1 ± 4.9 | 124.3 ± 4.9 | 0.0007 | 0.0005 | NS |
| | DBP* | 79.9 ± 3.4 | 81.6 ± 3.5 | 87.0 ± 3.5 | 0.009 | 0.02 | 0.05 |

*Only subjects not taking BP-lowering medication. NS = non significant

For independent replication, the Heredity And Phenotype Intervention (HAPI) Heart Study (10) was performed on an independently collected Amish sample for which subjects were not ascertained by a relative with diabetes (Table 1). HAPI subjects were genotyped using the Affymetrix GeneChip Human Mapping 500K platform. There were 66 SNPs located within 5 kb of STK39 with multiple SNPs in strong LD (R²>0.8) with the BP-associated SNPs on the 100K array. In addition, we genotyped SNPs rs6749447 and rs3754777 from the 100K array. While SNPs in multiple regions of this large gene were associated with BP, the strongest associations were those SNPs that are highly correlated with LD bin 1 SNPs from the 100K scan (P<0.001). In this younger and healthier cohort, associations to SBP achieved statistical significance. In this case, the mode of inheritance appeared recessive, while association was in the same direction and the effect size similar to that observed in the AFDS sample.

Although consistently heritable, BP can be a challenge to accurately assess because it varies greatly throughout the day and depends on the method of measurement. In the HAPI Heart study's protocol, BP was measured repeatedly both at home and in the clinic, using manual, automatic, and ambulatory BP monitors, at single time points and throughout a 24 hour period, while the subjects were on their usual versus standardized high and low salt diets. No HAPI subjects were taking BP-lowering medication during the study. Notably, Stk39 SNPs were associated with BP regardless of how BP was measured; the effect size is clinically relevant and estimated to be 5-9 mmHg SBP and 1-3 mmHg DBP (Table 3).

TABLE 3
Age and sex-adjusted effect size [95% confidence interval]
of rs6749447 (LD bin 1) from multiple
measurements ofBP in the AFDS and HAPI studies.

| Trait | SBP (mmHg) | DBP (mmHg) |
|---|---|---|
| AFDS | | |
| Home and clinic visits, manual | 5.7 [3.6 − 7.8] | 2.4 [1.1 − 3.7] |
| HAPI | | |
| Clinic, manual, regular diet | 9.2 [4.7-13.7] | 1.1 [−1.8-4.0] |
| Clinic, automatic, regular diet | 5.4 [1.1-9.7] | 2.4 [−0.1-4.8] |
| Home, manual, high salt | 9.4 [3.6-15.3] | 2.6 [−1.7-7.0] |
| Home, manual, low salt | 7.0 [1.5-12.5] | 2.2 [−1.7-6.1] |
| 24 hours ambulatory, high salt | 5.6 [0.6-10.6] | 1.3 [−1.8-4.5] |
| 24 hours ambulatory, low salt | 7.0 [2.7-11.2] | 2.1 [−0.8-5.0] |

Seeking additional replication and extension of the findings in non-Amish populations, publicly available GWA data from two studies that used the same genotyping platforms as the present invention was accessed: the Framingham Heart Study (FHS) and the Diabetes Genetics Initiative (DGI) with 1,345 and 3,082 subjects, respectively. Based on the criteria that the significance levels (P) must be <0.10 and the associations in the same direction as the Amish studies discussed herein, the Stk39 association with either SBP, DBP or EH status was replicated in both groups (Table 4 for LD bin 1 and 2 SNPs. All other association signals are provided in Tables 5 and 6A-6B). In Table 4, studies were grouped based on the Affymetrix platforms used to genotype. Minor allele frequencies (MAF) were study-specific. Only Ps<0.10 are shown. The associations to SBP in the FHS and to DP in the HAPI and DGI studies all have Ps≧0.10. Also, two studies with smaller sample sizes provided modest evidence for association between Stk39 and BP (Table 7). The same trend and effect size in a sample from another founder population, the Hutterites (N=575) was observed, but the difference did not reach significance level (LD bin 1 and 2 SNPs, P>0.10). The same is true when rs6749447 from LD bin 1 in GenNet Caucasian subjects was genotyped and analyzed (N=802, none taking medication). The effect size observed in Gen Net is based only on subjects not taking BP-lowering medication and is similar to what was observed in the expanded AFDS subjects (Table 2).

TABLE 4
Association results in OOA and non-Amish replication studies.

| LD | 100K | AFDS* | | | FHS** | | | 500K | HAPI*** | | DGI\hat{ } | | |
|---|---|---|---|---|---|---|---|---|---|---|---|---|---|
| bin | SNP ID | MAF | SBP | DBP | MAF | SBP | DBP | SNP ID | MAF | SBP | MAF | SBP | HTN |
| 1 | | | | | | | | rs6714609 | 0.24 | 0.0005 | 0.27 | 0.02 | 0.01 |
| | | | | | | | | rs6714707 | 0.21 | 0.0004 | 0.26 | 0.02 | 0.02 |
| | rs2278785 | 0.19 | 8 × 10⁻⁶ | 3 × 10⁻⁴ | 0.31 | | | rs7589259 | 0.22 | 0.0008 | 0.28 | | |
| | rs6749447 | 0.19 | 1 × 10⁻⁵ | 2 × 10⁻⁴ | 0.29 | 0.08 | 0.03 | rs6740492 | 0.22 | 0.0007 | 0.28 | | |
| | rs6734514 | 0.19 | 1 × 10⁻⁵ | 2 × 10⁻⁴ | 0.29 | 0.06 | 0.02 | rs4668040 | 0.22 | 0.0006 | 0.29 | | |
| | rs4668044 | 0.19 | 2 × 10⁻⁵ | 1 × 10⁻⁴ | 0.29 | 0.04 | 0.02 | rs13385577 | 0.22 | 0.0007 | 0.22 | 0.07 | 0.03 |
| | rs2063959 | 0.19 | 2 × 10⁻⁵ | 6 × 10⁻⁴ | 0.29 | 0.05 | 0.02 | rs10202854 | 0.22 | 0.0007 | 0.22 | 0.07 | 0.03 |
| | | | | | | | | rs4667570 | 0.22 | 0.0006 | 0.28 | | |
| | | | | | | | | rs4668046 | 0.22 | 0.0007 | 0.27 | | 0.05 |
| | | | | | | | | rs7605161 | 0.23 | 0.0008 | 0.29 | | |
| 2 | rs3769394 | 0.09 | 3 × 10⁻⁷ | 9 × 10⁻⁶ | 0.18 | 0.07 | 0.08 | | | | | | |
| | rs10497336 | 0.08 | 3 × 10⁻⁶ | 7 × 10⁻⁵ | 0.17 | | | rs1517329 | 0.12 | | 0.14 | 0.03 | |
| | rs3769392 | 0.10 | 4 × 10⁻⁶ | 4 × 10⁻⁵ | 0.19 | | | rs1400641 | 0.14 | 0.09 | 0.15 | 0.05 | |
| | rs3754781 | 0.10 | 3 × 10⁻⁷ | 1 × 10⁻⁵ | 0.17 | | | rs2063958 | 0.12 | | 0.14 | 0.03 | |
| | rs3754777 | 0.09 | 1 × 10⁻⁷ | 1 × 10⁻⁵ | 0.18 | | 0.09 | rs3769393 | 0.12 | | 0.15 | 0.02 | |
| | rs1400644 | 0.09 | 1 × 10⁻⁷ | 7 × 10⁻⁶ | 0.20 | | | rs10497337 | 0.12 | | 0.14 | 0.04 | |
| | rs1400645 | 0.09 | 1 × 10⁻⁷ | 1 × 10⁻⁵ | 0.20 | | | rs16855027 | 0.13 | | 0.14 | 0.03 | |
| | rs10497337 | 0.09 | 3 × 10⁻⁶ | 5 × 10⁻⁴ | 0.18 | | 0.07 | rs16855079 | 0.12 | | 0.14 | 0.02 | |
| | rs3769394 | 0.09 | 3 × 10⁻⁷ | 9 × 10⁻⁶ | 0.18 | | | | | | | | |

*Ps from additive models are presented.
**GEE Ps on examination 6 are presented.
***Ps from recessive models are presented.
\hat{ }Ps are based on linear regression after correction by genomic control.

TABLE 5
Association signals in FHS

| | | | DBP No. | | | | | | | | | | | | | | | |
|---|---|---|---|---|---|---|---|---|---|---|---|---|---|---|---|---|---|---|
| | LD | | 1 | | 2 | | 3 | | 4 | | 5 | | 6 | | 7 | | 17 | |
| Marker | bin | MAF | AS | MV | AS | MV | AS | MV | AS | MV | AS | MV | AS | MV | AS | MV | AS | MV |
| rs10497331 | | 0.11 | 0.51 | 0.5 | 0.14 | 0.14 | 0.06 | 0.04 | 0.4 | 0.28 | 0.79 | 0.56 | 0.16 | 0.12 | 0.58 | 0.66 | 0.16 | 0.11 |
| rs10497332 | | 0.39 | 0.97 | 0.77 | 0.93 | 0.93 | 0.15 | 0.24 | 0.14 | 0.19 | 0.31 | 0.32 | 0.44 | 0.42 | 0.84 | 0.67 | 0.47 | 0.72 |
| rs9287890 | | 0.13 | 0.41 | 0.39 | 0.58 | 0.62 | 0.34 | 0.3 | 0.93 | 0.95 | 0.88 | 0.9 | 0.25 | 0.22 | 0.93 | 0.83 | 0.46 | 0.4 |
| rs10497333 | | 0.48 | 0.96 | 0.75 | 0.92 | 0.76 | 0.19 | 0.36 | 0.58 | 0.91 | 0.57 | 0.8 | 0.69 | 0.61 | 0.57 | 0.3 | 0.7 | 0.88 |
| rs10497334 | | 0.13 | 0.72 | 0.71 | 0.64 | 0.67 | 0.49 | 0.44 | 1 | 0.97 | 0.99 | 0.99 | 0.29 | 0.26 | 0.99 | 0.91 | 0.58 | 0.52 |
| rs4667995 | | 0.39 | 0.78 | 0.53 | 0.91 | 0.91 | 0.24 | 0.34 | 0.25 | 0.36 | 0.44 | 0.48 | 0.35 | 0.32 | 0.75 | 0.61 | 0.68 | 0.97 |
| rs1448833 | | 0.5 | 0.5 | 0.72 | 0.95 | 0.79 | 0.07 | 0.06 | 0.34 | 0.35 | 0.91 | 0.89 | 0.43 | 0.44 | 0.51 | 0.32 | 0.62 | 0.74 |
| rs4667996 | | 0.48 | 0.76 | 0.99 | 0.69 | 0.66 | 0.11 | 0.15 | 0.42 | 0.56 | 0.69 | 0.83 | 0.64 | 0.59 | 0.4 | 0.21 | 0.67 | 0.94 |
| rs4667556 | | 0.23 | 0.12 | 0.14 | 0.01 | 0.01 | 0.3 | 0.23 | 0.08 | 0.04 | 0.07 | 0.08 | 0.005 | 0.003 | 0.21 | 0.13 | 0.02 | 0.008 |
| rs6728405 | | 0.45 | 0.06 | 0.11 | 0.18 | 0.18 | 0.33 | 0.51 | 0.08 | 0.19 | 0.04 | 0.07 | 0.08 | 0.08 | 0.05 | 0.09 | 0.03 | 0.06 |
| rs6745588 | | 0.32 | 0.4 | 0.58 | 0.81 | 0.68 | 0.64 | 0.93 | 0.45 | 0.99 | 0.39 | 0.53 | 0.64 | 0.56 | 0.33 | 0.57 | 0.56 | 0.97 |
| rs755844 | | 0.2 | 0.49 | 0.36 | 0.23 | 0.15 | 0.87 | 0.6 | 0.31 | 0.13 | 0.14 | 0.11 | 0.03 | 0.02 | 0.61 | 0.4 | 0.19 | 0.06 |
| rs10497335 | | 0.22 | 0.17 | 0.16 | 0.06 | 0.04 | 0.89 | 0.69 | 0.28 | 0.13 | 0.21 | 0.22 | 0.01 | 0.007 | 0.36 | 0.25 | 0.1 | 0.04 |
| rs2278785 | 1 | 0.31 | 0.78 | 0.85 | 0.35 | 0.59 | 0.27 | 0.39 | 0.72 | 0.51 | 0.79 | 0.52 | 0.15 | 0.14 | 0.3 | 0.33 | 0.74 | 0.89 |
| rs3769394 | 2 | 0.18 | 0.86 | 0.84 | 0.35 | 0.54 | 0.95 | 0.72 | 0.51 | 0.58 | 1 | 0.69 | 0.14 | 0.14 | 0.76 | 0.74 | 0.58 | 0.64 |
| rs10497336 | 2 | 0.17 | 0.74 | 0.69 | 0.27 | 0.34 | 0.97 | 0.86 | 0.58 | 0.58 | 0.79 | 0.56 | 0.18 | 0.18 | 0.43 | 0.42 | 0.72 | 0.72 |
| rs3769392 | 2 | 0.19 | 0.61 | 0.63 | 0.47 | 0.62 | 0.89 | 0.61 | 0.33 | 0.38 | 0.81 | 0.81 | 0.17 | 0.2 | 0.31 | 0.26 | 0.62 | 0.7 |
| rs3754781 | 2 | 0.17 | 0.78 | 0.95 | 0.23 | 0.59 | 0.59 | 0.97 | 0.27 | 0.47 | 0.94 | 0.54 | 0.13 | 0.21 | 0.84 | 0.64 | 0.42 | 0.73 |
| rs3754777 | 2 | 0.18 | 0.62 | 0.65 | 0.2 | 0.4 | 0.93 | 0.75 | 0.33 | 0.41 | 0.94 | 0.73 | 0.08 | 0.09 | 0.59 | 0.55 | 0.44 | 0.54 |
| rs1517343 | | 0.49 | 0.88 | 0.79 | 0.73 | 0.57 | 0.21 | 0.09 | 0.9 | 0.62 | 0.95 | 0.55 | 0.49 | 0.68 | 0.52 | 0.94 | 0.96 | 0.62 |
| rs1400644 | 2 | 0.2 | 0.63 | 0.45 | 0.23 | 0.24 | 0.56 | 0.53 | 0.97 | 0.91 | 0.68 | 0.55 | 0.16 | 0.14 | 0.69 | 0.8 | 0.78 | 0.62 |
| rs1400645 | 2 | 0.2 | 0.8 | 0.6 | 0.27 | 0.3 | 0.55 | 0.5 | 0.96 | 0.94 | 0.67 | 0.53 | 0.16 | 0.14 | 0.69 | 0.8 | 0.84 | 0.68 |
| rs10497337 | 2 | 0.18 | 0.55 | 0.53 | 0.22 | 0.37 | 0.92 | 0.65 | 0.38 | 0.48 | 0.99 | 0.75 | 0.09 | 0.09 | 0.68 | 0.66 | 0.46 | 0.51 |
| rs6740826 | | 0.11 | 0.39 | 0.45 | 0.33 | 0.37 | 0.04 | 0.03 | 0.48 | 0.39 | 1 | 0.75 | 0.41 | 0.32 | 0.95 | 0.94 | 0.47 | 0.46 |
| rs6749447 | 1 | 0.29 | 0.49 | 0.5 | 0.08 | 0.13 | 0.24 | 0.3 | 0.84 | 0.71 | 0.8 | 0.83 | 0.09 | 0.07 | 0.43 | 0.53 | 0.49 | 0.48 |
| rs6734514 | 1 | 0.29 | 0.4 | 0.38 | 0.10 | 0.16 | 0.18 | 0.23 | 0.94 | 0.9 | 0.92 | 0.93 | 0.09 | 0.07 | 0.55 | 0.64 | 0.39 | 0.39 |
| rs4668044 | 1 | 0.29 | 0.35 | 0.32 | 0.06 | 0.11 | 0.18 | 0.22 | 0.84 | 0.96 | 0.91 | 0.96 | 0.09 | 0.06 | 0.56 | 0.66 | 0.33 | 0.34 |
| rs2063959 | 1 | 0.29 | 0.32 | 0.28 | 0.07 | 0.11 | 0.23 | 0.28 | 0.9 | 0.75 | 0.99 | 0.93 | 0.08 | 0.05 | 0.56 | 0.66 | 0.36 | 0.36 |
| rs10497338 | | 0.48 | 0.64 | 0.63 | 0.87 | 0.82 | 0.44 | 0.42 | 0.85 | 0.85 | 0.94 | 0.85 | 0.41 | 0.43 | 0.66 | 0.83 | 1 | 0.97 |
| rs950535 | | 0.41 | 0.73 | 0.94 | 0.56 | 0.71 | 0.6 | 0.62 | 0.07 | 0.08 | 0.64 | 0.83 | 0.76 | 0.88 | 0.77 | 0.81 | 0.42 | 0.52 |

AS = adjusted for age and sex. MV = adjusted for multiple variables. SBP/DBP traits from examination 1, 2, 3, 4, 5, 6, 7 and long term averaged BP (17) are examined for replication to the Amish association findings in STK39. Ps > 0.10 are shown as empty boxes.

TABLES 6A-6B.
Additional STK39 SNPS that also demonstrated replication association
A.

| | | LD | HapMap | AFDS | | | FHS | | | | | |
|---|---|---|---|---|---|---|---|---|---|---|---|---|
| SNP ID. | location | bin | MAF | SBP | DBF | HTN | SBP2 | SBP6 | SBP17 | DBP2 | DBP6 | DBP17 |
| rs4667556 | 168663061 | A | 0.20 | 0.006 | 0.003 | 0.06 | 0.01 | 0.01 | 0.08 | 0.01 | 0.003 | 0.008 |
| rs755844 | 168681317 | | 0.20 | 0.006 | 0.004 | 0.06 | 0.08 | | | 0.04 | 0.02 | 0.06 |
| rs10497335 | 168681663 | | 0.20 | 0.006 | 0.004 | 0.06 | 0.03 | | | | 0.007 | 0.04 |

B.

| | | | | HAPI | | | | | | |
|---|---|---|---|---|---|---|---|---|---|---|
| | | LD | HapMap | SBP | SBP | DBP | DBP | DGI | | |
| SNP ID. | location | bin | MAF | Add | Dom | Add | Dom | SBP | DBP | HTN |
| rs1816977 | 168684033 | A | 0.19 | | | | | 0.03 | | |
| rs4668021 | 168684194 | | 0.19 | | | 0.06 | | 0.02 | | |
| rs2138753 | 168693548 | | 0.21 | | | 0.03 | 0.05 | 0.08 | | |
| rs13419175 | 168696185 | | 0.22 | | | | | 0.08 | | |
| rs1448831 | 168586040 | B | 0.34 | | 0.03 | | 0.05 | | 0.05 | |
| rs11685807 | 168593579 | | 0.34 | | 0.05 | | 0.05 | | 0.06 | |
| rs6718607 | 168594385 | | 0.36 | | 0.04 | | 0.06 | | | |
| rs4668002 | 168601200 | | 0.36 | | 0.03 | | 0.05 | | 0.03 | |
| rs6433032 | 168617066 | | 0.34 | | 0.04 | | 0.06 | | 0.05 | |
| rs4233815 | 168641089 | | 0.34 | | 0.04 | | 0.06 | | 0.05 | |
| rs6433027 | 168572606 | C | 0.44 | | 0.009 | | | 0.05 | | 0.03 |
| rs4668000 | 168573524 | | 0.44 | | | | | | | |
| rs4667548 | 168594440 | | 0.45 | | 0.007 | | 0.08 | | | 0.06 |
| rs9789702 | 168600664 | | 0.44 | | 0.007 | | 0.08 | 0.09 | | 0.06 |
| rs4667551 | 168612868 | | 0.44 | | 0.007 | | 0.07 | | | |
| rs10170500 | 168717811 | D | 0.17 | | 0.08 | | | | | 0.008 |
| rs3754776 | 168724417 | | 0.13 | | 0.09 | | | | | |
| rs6740826 | 168735801 | | 0.13 | | 0.08 | | | | | 0.02 |
| rs16855116 | 168768914 | | 0.13 | | | | | | | 0.04 |
| rs16855133 | 168777963 | | 0.13 | | 0.08 | | | | | 0.01 |

TABLE 7
Evidence of association in the GenNet Caucasian sample and in the Hutterites

| | LD | | | | | |
|---|---|---|---|---|---|---|
| | bin | SNP ID. | trait | Genotypic BP (Mean ± SD) | | |
| GenNet* | 1 | rs6749447 | | TT | GT | GG |
| | | (MAF = 0.20) | N. | 478 | 242 | 30 |
| | | | SBP | 118.4 ± 17.5 | 119.3 ± 17.1 | 122.1 ± 12.0 |
| | | | DBP | 75.4 ± 10.9 | 76.6 ± 10.9 | 76.4 ± 7.7 |
| Hutterite | 1 | rs6714609 | | GG | GA | AA |
| | | (MAF = 0.26) | N. | 340 | 226 | 43 |
| | | | SBP | 119.4 ± 15.4 | 120.6 ± 15.9 | 127.5 ± 17.6 |
| | | | DBP | 74.2 ± 9.7 | 74.9 ± 9.7 | 79.5 ± 9.8 |
| | | rs7589259 | | CC | CT | TT |
| | | (MAF = 0.25) | N. | 352 | 219 | 50 |
| | | | SBP | 119.4 ± 15.4 | 121.0 ± 16.1 | 126.2 ± 17.0 |
| | | | DBP | 74.2 ± 9.8 | 75.3 ± 9.7 | 77.7 ± 10.3 |
| | | rs4668046 | | AA | AG | GG |
| | | (MAF = 0.27) | N. | 322 | 210 | 52 |
| | | | SBP | 119.8 ± 15.2 | 120.3 ± 16.3 | 125.8 ± 17.2 |
| | | | DBP | 74.5 ± 9.4 | 75.0 ± 9.9 | 77.5 ± 9.9 |
| | 2 | rs1517329 | | TT | TC | CC |
| | | (MAF = 0.14) | N. | 440 | 129 | 17 |
| | | | SBP | 120.4 ± 16.0 | 119.6 ± 14.9 | 130.8 ± 18.3 |
| | | | DBP | 74.3 ± 9.5 | 76.5 ± 9.9 | 80.0 ± 8.1 |
| | | rs2063958 | | AA | AG | GG |
| | | (MAF = 0.14) | N. | 474 | 136 | 18 |
| | | | SBP | 120.4 ± 16.0 | 119.7 ± 14.6 | 129.3 ± 18.8 |
| | | | DBP | 74.3 ± 9.7 | 76.4 ± 10.0 | 79.9 ± 7.9 |

*Only Caucasian subjects who were not taking hypertension medication.

Tables 6A-6B are SNPS tagged by the Affymetrix 100K and 500K array, respectively. Only Ps<0.10 are shown (empty boxes are either SNPs that did not reach significance level or deleted during study specific QC procedure). SNPs within LD bins have similar MAFs and are highly correlated with each other (R²>0.8). For FHS, Ps from Sbp and DBP measured during examination 2, 6 and averaged using all data from examination 1 to 7 (denoted by FHS as “17”) are shown.

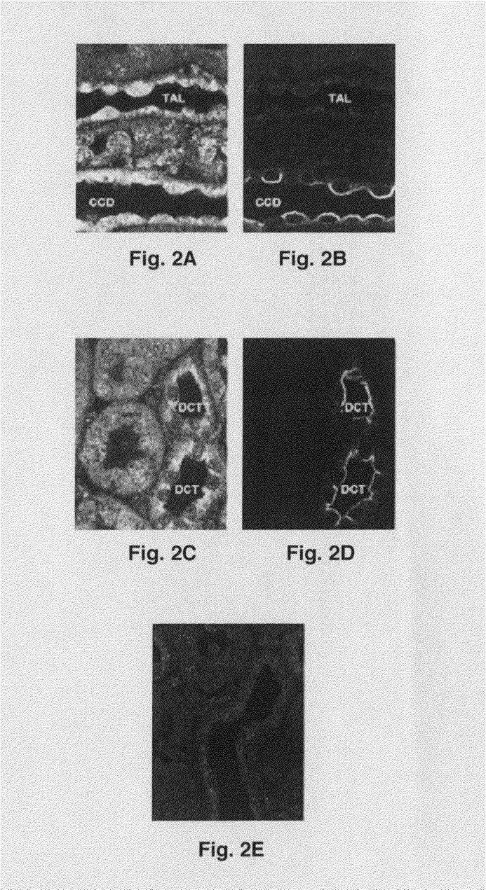

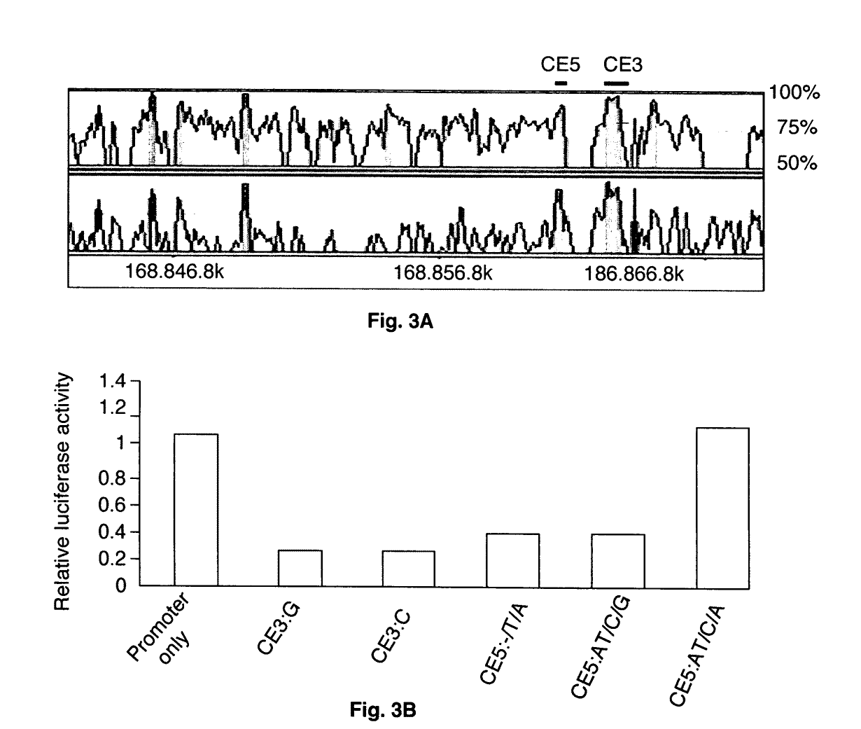

The identification of Stk39 as a susceptibility gene for EH raises the intriguing possibility that SPAK controls BP by regulating renal ion transport. Consistent with this idea, recent in vitro studies demonstrate that SPAK binds to and phosphorylates two Na⁺-dependent cation chloride cotransporters that mediate renal NaCl reabsorption, NKCC2 and NCC. To explore whether SPAK has the capacity to regulate these transporters in vivo, the renal expression pattern of SPAK was determined by immunofluorescence microscopy of rat kidney sections. SPAK immunostaining was observed at the site of NKCC2 expression, the thick ascending limb of the Loop of Henle (TAL) (FIG. 2A), as well as at the predominant site of NCC expression, the distal convoluted tubule (DCT) (FIG. 2C). In addition, robust SPAK expression was observed in cortical collecting duct (CCD) (FIG. 2A). The specificity of the SPAK antibody was confirmed by peptide competition (FIG. 2E). The immunolocalization of SPAK to these nephron segments provides evidence that genetic variants of Stk39 may alter BP and urinary electrolyte excretion by influencing the activity of NKCC2, NCC, or other mediators of ion flux in the distal nephron.

The associated SNPs in LD bin 1 and 2 are located in a 76 kb region that spans intron 1 to 9. All the exons and evolutionarily conserved non-coding elements in the associated region were sequenced. It was observed that several associated SNPs were located in highly conserved intronic elements that may influence SPAK expression. It remains to be determined how these SNPs affect SPAK transcription, but a hypothesis is that a change in the SPAK abundance may alter the phosphorylation state of its downstream substrates, NKCC2 or NCC. Changes in SPAK expression may also affect the activity of binding partners that participate in SPAK-related signaling processes, such as the WNK kinases.

In addition to SNPs in LD bins 1 and 2, there are other STK39 SNPs reproducibly associated with BP levels (P<0.05 in two or more studies, Table 6). For example, a group of SNPs located in intron 10 are also associated with BP in AFDS, FHS, HAPI and DGI. Besides renal electrolyte transport, SPAK has been linked to functions such as cytoskeleton rearrangement, cell differentiation, transformation, and proliferation. In fact, in a subset of Amish subjects with extensive metabolic syndrome-related phenotypes, the BP-associated Stk39 SNPs were modestly associated with fasting glucose, insulin response to glucose, and plasma triglyceride levels.

The initial association discussed herein based on the GWA scanning set was significant at genomewide level even if one applies Bonferroni correction adjusting for the number of SNPs analyzed (0.0000001×82,485=0.008). This approach is overly conservative because over 40,000 SNPs on the 100K arrays are highly correlated with one or more SNP. In summary, the present invention establishes that SNPs within the STK39 gene are strongly associated with EH. The relevance of Stk39 as a EH susceptibility gene is valid across multiple populations. Although these studies were performed within specific cohorts, the reproducibility across populations provides compelling evidence that Stk39 expression may be altered in subsets of patients with EH. Thus, these findings suggest that Stk39 genotype might be a predictor of EH patients more likely to respond to salt-reduction and known diuretics as a measure to control BP and SPAK itself might be an ideal target for novel antihypertensive drug therapies.

As discussed supra, there is no reliable way available currently to predict the most suitable hypertension medication for individual patients, when to reduce salt in the diet and how to avoid undesirable side effects related to hypertension medication including but not limited to elevated glucose. Hence, the findings of the present invention lead to novel clinical applications for STK39 including but not limited to the following: First, as a novel target for novel hypertension medication. Stk39 phosphorylates and regulates 2 cation-transporters, Na⁺—Cl⁻ co-transporter (NCC) and Na⁺—K⁺-2Cl⁻ co-transporter (NKCC2). NCC and NKCC2 are sensitive to different types of diuretics, thiazide and bumetanide, respectively. Therefore, Stk39 may be amenable to pharmacologic intervention and is a target for the development of new hypertension and hypertension-related medication.

Second, as a marker to determine hypertensive patient who is salt-sensitive and more likely to benefit from low salt diet. When salt intake is increased, only certain individuals respond with higher blood pressure and are called “salt sensitive”. Similarly, only some hypertensive patients' blood pressure can be effectively controlled by reducing salt intake. The result presented herein indicate that individuals with certain Stk39 genotypes are more likely to benefit from low-salt diet.

Third, as a marker to determine the class of existing hypertensive medication more likely to be effective for a particular patient. There is no way to predict how an individual will respond to diuretics. There is a general belief that African-Americans are more likely to respond to diuretics. Presently, in the absence of a genetic marker, perceived or self-reported “racial” status is used as a surrogate. The data presented herein will provide a genetic marker to indentify hypertensive patients, who are more likely to benefit from salt reduction and certain types of blood pressure lowering medications.

Fourth, as a marker for hypertension medication-related adverse effect. It is known that medication intended to reduce blood pressure sometime causes patients to develop type 2 diabetes or become insulin resistant. In addition to baseline blood pressure, Stk39 genotypes are also associated with fasting glucose level, insulin response to glucose challenge and other traits related to metabolic syndrome. Thus, Stk39 can be used to predict subgroup of hypertensive patients that is more likely to experience adverse effect due to hypertension medication. This information can be considered before a particular blood pressure lowering medication is prescribed.

The concentration of the hypertension medication is known in the art. If the concentration is unknown as in the case of a potential hypertension medication, then one of skill in the art can determine such concentrations by performing experiments that are routine in the art

Treatment methods will involve treating hypertension in an individual with an pharmacologically effective amount of an anti-hypertension medication, low salt diet or a combination thereof. A pharmacologically effective amount is described, generally, as that amount sufficient to detectably and repeatedly prevent, ameliorate, reduce, minimize or limit the detection of one or more variants of Stk39 and extent of a hypertension. The pharmacologically effective amount of the hypertension medication to be used are those amounts effective to produce beneficial results, particularly with respect to preventing hypertension or hypertension-related adverse effects.

The existing or potential anti-hypertensive drug may be administered either alone or in combination with another drug or a compound. Such drug or compound may be a compound that inhibits the single nucleotide polymorphism in Stk39 or a protein encoded by this polymorphism may be administered concurrently or sequentially with the hypertension medication. The effect of co-administration with the anti-hypertensive drug is to lower the dosage of the drug or the compound normally required that is known to have at least a minimal pharmacological or therapeutic effect against the disease that is being treated. Concomitantly, toxicity of the drug or the compound to normal cells, tissues and organs is reduced without reducing, ameliorating, eliminating or otherwise interfering with any cytotoxic, cytostatic, apoptotic or other killing or inhibitory therapeutic effect of the drug or compound.

The anti-hypertensive drug and the drug or compound discussed herein may be administered independently, either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant. Dosage formulations of the composition described herein may comprise conventional non-toxic, physiologically or pharmaceutically acceptable carriers or vehicles suitable for the method of administration.

The anti-hypertensive drug and the drug or compound discussed herein may be administered independently one or more times to achieve, maintain or improve upon a therapeutic effect. It is well within the skill of an artisan to determine dosage or whether a suitable dosage of either or both of the anti-hypertensive drug or compound and the drug, or compound comprises a single administered dose or multiple administered doses.

As is well known in the art, a specific dose level of such an existing or potential anti-hypertensive drug and compound for any particular patient depends upon a variety of factors including the activity of the specific compound employed, the age, body weight, general health, sex, diet, time of administration, route of administration, rate of excretion, drug combination, and the severity of the particular disease undergoing therapy.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Study Subjects and Phenotype

### Amish Family Diabetes Study (AFDS)

Recruitment for the AFDS was initiated in 1995 with the goal of identifying susceptibility genes for type 2 diabetes (T2DM) and related traits in the Old Order Amish. Details of the AFDS design, recruitment, and pedigree structure are well known.

Briefly, study subjects received an extensive interview regarding their personal medical history and family history of diabetes and related diseases. SBP/DBP were obtained in duplicate with the use of a standard sphygmomanometer after the patient has been sitting for ≧5 minutes and were recorded to the nearest 1 mmHg. Heritability of BP levels and prevalence of EH (SBP≧140 mm Hg or DBP≧90 mm Hg or current use of antihypertensive medications) were determined and are comparable between the Amish population and a representative sample of the overall white population in the United States. From the entire AFDS, 551 subjects were selected to include 124, 132, and 295 subjects with T2DM, impaired glucose tolerance, and normal glucose tolerance, respectively and genotyped using Affymetrix 100K set¹⁷. The entire AFDS (n=1,093) was subsequently genotyped to confirm the top association findings (Table 1A).

### Hereditary and Phenotype Intervention (HAPI) Heart Study

The HAPI Heart Study was initiated in 2002 to measure the cardiovascular response to four short-term interventions affecting cardiovascular health and to identify the genetic and environmental determinants of these responses. The 4 interventions were 1) triglyceride excursion response to a high fat meal, 2) BP and endothelial function response to the cold pressor test, 3) BP response to a high and low salt diet, and 4) platelet reactivity response to aspirin.

A short summary of BP measurements obtained during the course of the study, as presented in Table 3, is given below. Prior to any intervention, baseline BP was obtained manually in the sitting position three times after the subject had been sitting quietly for 5 minutes at the Amish Research Clinic (clinic, manual, regular diet). On the same day, a series of BP readings (taken every 5 minutes over a 20-50 minute period) were also taken prior to the administration of cold pressor challenge using an automatic BP monitor (Accutorr Plus, Datascope). The baseline BP for response to cold pressor challenge was calculated using the last 2 readings once the BP had stabilized (clinic, automatic, regular diet).

Multiple BP measurements were obtained during the high/low salt interventions, which consisted of 6 days (Monday to Saturday) of an isocaloric high Na⁺ diet (280 meq/day) followed by an 8 day wash out period and then 6 days of an isocaloric low Na⁺ diet (40 meq/day). Both diets contained 140 meq K⁺/day. On the fifth day of each diet, study nurses traveled to the participant's home and measured resting BP manually in triplicate (home, manual, high/low salt). BP was also measured using an ambulatory BP monitor (SpacLabs, Inc.) for 24 hours on the last day of each of the diets and these values were averaged to obtain a single measurement of BP for that day (24 hours ambulatory, high/low salt).

### Framingham Heart Study (FHS)

Recruitment of men and women from the town of Framingham, Mass. began in 1948 with the purpose of investigating the causes of cardiovascular disease (CVD) and related traits. FHS investigators have recently published results of a 100K GWA study based on 1,345 subjects (310 pedigrees), 17 phenotypes, and the Affymetrix GeneChip® Mapping 100K Array. All associations are available through dbGaP.

Phenotypic characteristics of these subjects vary from examination to examination and the baseline data at examination 1 has been published. The use of EH medication was accounted for in the analysis. After genotyping quality control, 70,987 autosomal SNPs with MAF≧0.10, genotype call rate≧0.80, and HWE p≧0.001 were analyzed. For replication of the STK39 signals, SBP and DBP were used at examinations 1 to 7, and the average of SBP/DBP from available examinations 1 to 7. Results from both family-based association test (FBAT) and generalized estimating equations (GEE) using residuals obtained from regression models that are specific for the examination and accounted for age, sex, and BMI are available online. FBAT results are presented here for the purpose of replication and to determine the direction of effect.

### The Diabetes Genetics Initiative (DGI)

The DGI performed a GWA study in 1,464 type 2 diabetes patients and 1,467 controls to search for genetic variants that influence risk of diabetes and related traits, such as glucose, obesity, lipids, and BF. The subjects were Scandinavian individuals with ancestry from Finland and Sweden and the controls were either geographically matched with the cases or selected from discordant sibling pairs.

For the analysis of BP related traits, cases and controls were combined. The number of subjects analyzed for SBP, DBP, and EH status were 2,895, 1,247, and 3,082, respectively. The sample size for DBP was considerably smaller since subjects over age 60 were excluded from the analysis. Linear regression and logistic regression were used to test the association between genotypes and either blood pressure residual or EH status, respectively. Raw values of blood pressure were adjusted for EH medication use (28% of participants) prior to analysis as described (Levy et al., Hypertension 36, 477-83 (2000)). Overall, 386,731 SNPs passed genotype quality control based on the following criteria: MAF≧0.05, genotype call rate≧0.95, and HWE P≧10⁻⁶. Details of the phenotypic characteristics, analysis strategy, and association levels of all the SNPs are available online. For replication, Ps after correction by genomic control were used and regression coefficients (labeled as BETA) were used to determine the direction of association.

### Hutterites

The Hutterites are a religious isolate that originated in the Tyrolean Alps during the 1500s. In the 1870s, ˜900 Hutterites moved to what is now South Dakota and are now living in >350 communal farms in northern US and Canada. Because of their communal lifestyles, Hutterites share a relatively uniform environment. The 575 subjects in this study are descendants of 64 ancestors, and related to each other in a 13-generation pedigree. To test for effects of each SNP on SBP or DBP, the general two-allele model test of association was used in the entire pedigree, keeping all inbreeding loops intact, as described (Ober, C et al., Am J Hum Genet 69, 1068-79 (2001)).

### GenNet

DNA samples used in this study were collected through the GenNet network of the NHLBI Family Blood Pressure Program. Probands were those between the ages of 18 and 50 years with BP in the upper 25% of the age-/gender-specific BP distribution. Overall 33% of the study subjects were clinically hypertensive either with SBP≧140, DBP≧90 mmHg, or were on EH medication at the time of the study. The average systolic and diastolic readings from 2 manual readings using a standard mercury sphygmomanometer were used for analysis. Eight hundred and two Caucasian subjects from Tecumseh, Mich., who were not taking BP-lowering medication at the time of the study were analyzed.

### Example 2

### Genotyping and Genotype Data Quality Control

For the initial genome-wide association scan conducted in the AFDS samples, genomic DNA from leukocytes were genotyped using the Affymetrix GeneChip® Mapping 100K Array set. Genotyping protocol and quality control procedures used to identify and remove poor-quality 100K data. Individual SNPs with genotype call rates<90%, SNPs not mapped to a unique position, monomorphic SNPs and SNPs with MAF<5%, and those deviating from Hardy-Weinberg equilibrium (HWE) (P<0.001) were removed. Eighty-two thousand four hundred and eighty-five autosomal SNPs (median and mean inter-SNP distance=11.3 kb and 29 kb, respectively) were analyzed. The genotype quality control procedure and concordance rates across 3 genotyping platforms are detailed in Table 5.

For HAPI Heart study subjects, GWAS was carried out using the Affymetrix GeneChip® Human Mapping 500K Array set. The 500K array set consists of two microarray chips (Nspl and Styl), which collectively include a total of 500,568 SNPs. Total genomic DNA (250 ng) was digested with Nspl or Styl and processed according to the Affymetrix protocol. The GeneChip Genotyping Analysis Software (GTYPE 4.0) was used for automated genotype calling as part of the GeneChip Operating Software (GCOS) platform. The GTYPE-generated chip files were re-analyzed using the BRLMM genotype calling algorithm which provided improved call accuracy compared with the DM algorithm (http://www.affymetrix.com). A confidence threshold, a measure of the call quality, of 0.5 was used for this analysis. As an initial quality control measure, BRLMM-generated chip files with genotype call rates<93% for both microarrays were regenotyped before further analysis. The resulting mean genotype call rate was 98.3%. Uninformative SNPs that had MAF<2% in the overall sample (n=98,806) were removed from further analyses. Finally, of the 369,241 informative SNPs that passed quality control and HWE checks (using a cut-point of P<0.001), 66 SNPs were located within 5 kb of STK39 and were analyzed for this study.

Additional genotyping of STK39 SNPs were performed using 5′ nuclease-based assay (TaqMan, ABI). Data quality of SNP genotypes was established by 3 methods: by reproducibility of control DNA samples; by expected Mendelian inheritance of alleles within a family (PedCheck; 21); and by HWE tests (P>0.05). For the latter, all unrelated individuals in the study analyzed using the Haploview analysis package.

### Example 3

### Immunolocalization of SPAK

Kidneys from adult Sprague-Dawley rats were fixed by retrograde perfusion and embedded in paraffin. Once sections (3 μm) were picked up on cover slips, heat-induced target retrieval using a citrate buffer (pH 8) was used to unmask epitopes. Sections were then washed and incubated overnight with primary rabbit antibodies to SPAK (Abgent, San Diego, Calif.) at 5 ug/ml at 4° C. followed by secondary antibodies as previously described (Coleman, R A et al., J Histochem Cytochem 54, 817-27 (2006)).

### Example 4

### Statistical Analyses

Because of the highly complex pedigree structure of the Amish families, the initial scan adopted a regression approach that ignored relatedness between subjects. The 2,000 association signals most highly associated with BP were re-analyzed using a computationally intensive variance component approach that adjusted for the relationship among subjects. The initial GWA was performed in a sample of 551 participants from the AFDS. Analysis consisted of estimating genotypic differences in mean age and sex adjusted residuals. Residual SBP and DBP were estimated for each individual using linear regression accounting for age, age and sex. Genotypic differences of residual values were then tested using 1 degree of freedom tests. This analysis was performed in HelixTree (Golden Helix, Bozeman, Mont.).

Since individuals in the Amish samples are related, it has been shown that this methodology returns valid point estimates but inflated variance around those estimates resulting in a predictable increase in type 1 error rates (23). In order to account for family structure, significant models were further assessed using variance components. Here trait values were modeled as effects of age, age, sex, genotype, and each pairwise relationship between individuals in the sample. This analysis was performed using the SOLAR software package (Southwest Foundation for Biomedical Research, San Antonio Tex.). All P-values reported were after correcting for pedigree structure. Pairwise R² values between SNPs were calculated using Haploview. LD bin assignment was based on HapMap CEU genotypes, which provided LD structure of the STK39 region that is nearly identical to that observed in the Amish.

When evaluating association signals from non-Amish studies, study-specific definition of EH and adjustment of medication were used and the association analyses provided online were considered. Directionality of the association was determined by either FBAT statistics of beta. The significance levels some studies where effect size could not be determined are provided.

What is claimed is:

1. A method of determining susceptibility of an individual to developing essential hypertension, comprising:
detecting at least one single nucleotide polymorphism in the Stk39 gene in a sample from the individual, wherein the presence of said single nucleotide polymorphism in the sample indicates that said individual is susceptible to develop essential hypertension.

2. The method of claim 1, wherein said single nucleotide polymorphism(s) is located in intron 10 of the Stk39 gene.

3. The method of claim 1, wherein said single nucleotide polymorphism is rs2278785, rs3769394, rs10497336, rs3769392, rs3754781, rs3754777, rs1400644, rs1400645, rs10497337, rs6749447, rs6734514, rs4668044, rs2063959, rs10497331, rs10497332, rs9287890, rs10497333, rs10497334, rs4667995, rs1448833, rs4667556, rs6728405, rs6745588, rs755844, rs10497335, rs1517343, rs6740826, rs10497338, rs950535, rs4667996, rs1816977, rs4668021, rs2138753, rs13419175, rs1448831, rs11685807, rs6718607, rs4668002, rs6433032, rs4233815, rs6433027, rs4668000, rs4667548, rs9789702, rs4667551, rs10170500, rs3754776, rs6740826, rs16855116, rs1685513, rs6714609, rs7589259, rs4668046, rs1517329, rs2063958, rs4668040, rs13385577, rs10202854, rs4667570, rs7605161, rs1400641, rs3769393, rs16855027, rs16855079, rs12692877, or rs35929607.

4. The method of claim 1, wherein said detection is at the nucleic acid or protein level.

5. The method of claim 4, wherein the detection at the nucleic acid level is by a method selected from the group consisting of DNA microarray, PCR assay and FISH.

6. The method of claim 4, wherein the detection at the protein level is by a method selected from the group consisting of ELISA, Western blot, immunohistochemistry and HPLC.

7. The method of claim 1, wherein said biological sample is selected from the group consisting of serum, urine, skin biopsy, and buccal swab.

8. The method of claim 1, wherein presence of said single nucleotide polymorphism in the sample indicates that said individual can be treated with an anti-hypertensive drug with a decreased adverse effect on glucose homeostasis and/or a low salt diet with a decreased adverse effect on glucose homeostasis.

9. The method of claim 8, wherein said anti-hypertensive drug is selected from the group consisting of a diuretic, an angiotensin converting enzyme inhibitor, angiotensin II receptor antagonist, an alpha blocker, a beta blocker, a calcium channel blocker and a direct renin inhibitor.

10. A method of treating an individual with essential hypertension, comprising:
administering pharmacologically effective amounts of a compound that inhibits at least one single nucleotide polymorphism in Stk39 at the nucleic acid level and/or inhibits at least one protein encoded by a single nucleotide polymorphism in Stk39 to the individual, thereby treating the individual with essential hypertension.

11. The method of claim 10, further comprising:
reducing sodium intake in said individual.

12. The method of claim 10, wherein administration of said compound results in a decreased adverse effect on the glucose homeostasis of said individual.

13. The method of claim 10, wherein said single nucleotide polymorphism(s) is located in intron 10 of the Stk39 gene.

14. The method of claim 10, wherein said single nucleotide polymorphism is rs2278785, rs3769394, rs10497336, rs3769392, rs3754781, rs3754777, rs1400644, rs1400645, rs10497337, rs6749447, rs6734514, rs4668044, rs2063959, rs10497331, rs10497332, rs9287890, rs10497333, rs10497334, rs4667995, rs1448833, rs4667556, rs6728405, rs6745588, rs755844, rs10497335, rs1517343, rs6740826, rs10497338, rs950535, rs4667996, rs1816977, rs4668021, rs2138753, rs13419175, rs1448831, rs11685807 rs6718607, rs4668002, rs6433032, rs4233815, rs6433027, rs4668000, rs4667548, rs9789702, rs4667551, rs10170500, rs3754776, rs6740826, rs16855116, rs1685513, rs6714609, rs7589259, rs4668046, rs1517329, rs2063958, rs4668040, rs13385577, rs10202854, rs4667570, rs7605161, rs1400641, rs3769393, rs16855027, rs16855079, rs12692877, or rs35929607.

15. A method of treating an individual with essential hypertension, comprising:
administering pharmacologically effective amounts of an anti-hypertensive drug to the individual, wherein said individual has at least one single nucleotide polymorphism in Stk39 gene, thereby treating the individual with essential hypertension.

16. The method of claim 15, further comprising:
reducing sodium intake in said individual.

17. The method of claim 15, wherein administration of said anti-hypertensive drug results in a decreased adverse effect on glucose homeostasis of said individual.

18. The method of claim 15, wherein the anti-hypertensive drug is selected from the group consisting of a diuretic, an angiotensin converting enzyme inhibitor, an angiotensin II receptor antagonist, an alpha blocker, a beta blocker, a calcium channel blocker and a direct renin inhibitor.

19. The method of claim 15, wherein said single nucleotide polymorphism(s) is located in intron 10 of the Stk39 gene.

20. The method of claim 15, wherein said single nucleotide polymorphism is rs2278785, rs3769394, rs10497336, rs3769392, rs3754781, rs3754777, rs1400644, rs1400645, rs10497337, rs6749447, rs6734514, rs4668044, rs2063959, rs10497331, rs10497332, rs9287890, rs10497333, rs10497334, rs4667995, rs1448833, rs4667556, rs6728405, rs6745588, rs755844, rs10497335, rs1517343, rs6740826, rs10497338, rs950535, rs4667996, rs1816977, rs4668021, rs2138753, rs13419175, rs1448831, rs11685807, rs6718607, rs4668002, rs6433032, rs4233815, rs6433027, rs4668000, rs4667548, rs9789702, rs4667551, rs10170500, rs3754776, rs6740826, rs16855116, rs1685513, rs6714609, rs7589259, rs4668046, rs1517329, rs2063958, rs4668040, rs13385577, rs10202854, rs4667570, rs7605161, rs1400641, rs3769393, rs16855027, rs16855079, rs12692877, or rs35929607.
